(19) United States
(12) Patent Application Publication
(10) Pub. No.: US 2019/0106719 A1
(43) Pub. Date: Apr. 11, 2019

(54) RECOMBINANT YEAST AND METHOD FOR PRODUCING ETHANOL USING THE SAME

(71) Applicant: TOYOTA JIDOSHA KABUSHIKI KAISHA, Toyota-shi (JP)

(72) Inventors: Toru ONISHI, Toyota-shi (JP); Nobuki TADA, Nisshin-shi (JP); Junji ITO, Nisshin-shi (JP); Rie HIRAO, Handa-shi (JP)

(73) Assignee: TOYOTA JIDOSHA KABUSHIKI KAISHA, Toyota-shi (JP)

(21) Appl. No.: 16/154,835

(22) Filed: Oct. 9, 2018

(30) Foreign Application Priority Data
Oct. 11, 2017 (JP) 2017-197752

(51) Int. Cl. C12P 7/10; C12N 9/92; C12R 1/865

(52) U.S. Cl. CPC C12P 7/10; C12N 9/92; C12R 1/865; C12Y 503/01018; C12P 2203/00; C12Y 503/01009; C12Y 503/01005

(57) ABSTRACT

This invention is aimed at improving an ethanol fermentation ability of a recombinant yeast strain having an ability of assimilating pentose, such as xylose or arabinose. The recombinant yeast strain haying an ability of assimilating pentose is obtained by lowering activity of a gene involved in upstream of glyceraldehyde-3-phosphate in the Embden-Meyerhof pathway.

### CROSS REFERENCE TO RELATED APPLICATIONS

The present application claims priority from Japanese patent application JP 2017-197752 filed on Oct. 11, 2017, the content of which is hereby incorporated by reference into this application.

### BACKGROUND

### Technical Field

The present disclosure relates to a recombinant yeast strain having xylose-metabolizing ability and a method for producing ethanol using the same.

### Background Art

A cellulosic biomass is an effective starting material for a useful alcohol, such as ethanol, or an organic acid. In order to increase the amount of ethanol produced with the use of a cellulosic biomass, yeast strains capable of utilizing xylose, which is pentose, as a substrate have been developed. For example, JP 2009-195220 A discloses a recombinant yeast strain resulting from incorporation of a xylose reductase (XR) gene and a xylitol dehydrogenase (XDH) gene derived from Pichia stipitis into its chromosome. Also, a report has been made concerning a xylose-assimilating yeast strain into which a xylose isomerase (XI) gene (derived from the intestinal protozoa of termites) has been introduced (IP 2011-147445 A). When both XR and XDH are introduced or when XI is introduced, xylulose is generated in the xylose-assimilating pathway, and xylulose is converted into xylulose 5-phosphate with the aid of xylulokinase. Xylulose 5-phosphate is metabolized in the pentose phosphate pathway and is then converted into glyceraldehyde-3-phosphate. Glyceraldehyde-3-phosphate enters the glycolytic pathway and ethanol is generated in the end.

The glycolytic pathway is a pathway by which glucose is metabolized, and a major pathway in a yeast strain is the Embden-Meyerhof pathway. The Embden-Meyerhof pathway (it is occasionally referred to as “the Embden-Meyerhof-Parnas pathway”) contains, as an intermediate metabolite, glyceraldehyde-3-phosphate. In the xylose-assimilating pathway and the pentose phosphate pathway, glyceraldehyde-3-phosphate metabolized from xylose enters the glycolytic pathway, and ethanol is generated in the end.

In a yeast strain into which no xylose-assimilating pathway has been introduced, disruption of a gene in the Embden-Meyerhof pathway leads to direct inhibition of glucose metabolism. In a medium comprising, as a major sugar source, glucose, accordingly, growth or ethanol fermentation is inhibited (see FIG. 1 of Applied Microbiology and Biotechnology 2013, 97, 3569-3577 concerning disruption of the phosphofructokinase (PFK1) gene). It is also reported that a yeast strain into Which a xylose-assimilating pathway has been introduced would not grow in a medium containing xylose as a carbon source if the gene in the Embden-Meyerhof pathway (i.e., the glucose phosphate isomerase (PGI1) gene) has been disrupted (Yeast, 2011. 28, 645-660 and FEMS yeast research 8, 2008, 217-224).

Meanwhile, glycerin is a representative by-product of ethanol production in a yeast strain, and it is important to reduce glycerin in order to improve an ethanol yield. To date, attempts of improving ethanol productivity by disrupting genes associated with pathways of producing glycerin from glyceraldehyde-3-phosphate; i.e., GPD1, GPD2, GPP1, and GPP2, lowering the expression levels thereof, and attenuating activity thereof have been reported. However, it is also reported that direct attenuation of such pathways would cause side effects, such as lowered rate of ethanol production (Appl. Environ. Microbiol., 2011, 77, 5857-5867 and Appl. Environ. Microbiol., 2013, 79, 3273-3281).

### SUMMARY

Under the above circumstances, in particular, the present disclosure relates to improving ethanol fermentation ability of a recombinant yeast strain having an ability of assimilating pentose, such as xylose or arabinose.

We have conducted concentrated studies aimed at improving ethanol fermentation ability of a recombinant yeast strain having an ability of assimilating pentose, such as xylose or arabinose. As a result, we discovered that ethanol fermentation ability of the recombinant yeast strain of interest could be improved to a significant extent by lowering activity of a gene involved in upstream of glyceraldehyde-3-phosphate in the Embden-Meyerhof pathway. This has led to the completion of the present disclosure.

The present disclosure includes the following.

(1) A recombinant yeast strain having an ability of assimilating pentose, which is obtained by lowering activity of a gene involved in upstream of glyceraldehyde-3-phosphate in the Embden-Meyerhof pathway.
(2) The recombinant yeast strain according to (1), wherein the gene involved in upstream of glyceraldehyde-3-phosphate in the Embden-Meyerhof pathway is at least one gene selected from the group consisting of the hexokinase gene, the glucose phosphate isomerase gene, the phosphofructokinase gene, and the fructose bisphosphate aldolase gene.
(3) The recombinant yeast strain according to (1), wherein the amount of glycerin production is reduced, compared with a yeast strain that retains the gene activity.
(4) The recombinant yeast strain according to (1), wherein the pentose is xylose and/or arabinose.
(5) The recombinant yeast strain according to (1), which comprises the xylose isomerase gene introduced thereinto and has the xylose-assimilating ability.
(6) The recombinant yeast strain according to (5), which further comprises a xylulokinase gene introduced thereinto.
(7) The recombinant yeast strain according to (1), which comprises a gene encoding an enzyme selected from a group of enzymes constituting a non-oxidative process in the pentose phosphate pathway introduced thereinto.
(8) The recombinant yeast strain according to (7), wherein the group of enzymes constituting a non-oxidative process in the pentose phosphate pathway includes ribose-5-phosphate isomerase, ribulose-5-phosphate-3-epimerase, transketolase, and transaldolase.
(9) A method for producing ethanol comprising a step of ethanol fermentation by culturing the recombinant yeast strain according to any of (1) to (8) in a medium containing assimilable pentose.
(10) The method for producing ethanol according to (9), wherein the medium contains cellulose and the ethanol fermentation proceeds simultaneously at least with cellulose saccharification.

### Effects

The recombinant yeast strain of the present disclosure has an ability of assimilating pentose, and has ethanol fermentation ability significantly improved compared with the yeast strain that maintains activity of a gene involved in upstream of glyceraldehyde-3-phosphate in the Embden-Meyerhof pathway. With the use of the recombinant yeast strain and the method of producing ethanol utilizing such yeast strain of the present disclosure, excellent ethanol yield can be achieved.

### BRIEF DESCRIPTION OF THE DRAWINGS

FIG. 1 is a metabolic map schematically showing a. process of ethanol fermentation from glucose and xylose.

### DETAILED DESCRIPTION

Hereafter, the present disclosure is described in greater detail with reference to the drawings and the examples.

### [Recombinant Yeast Strain]

The recombinant yeast strain of the present disclosure has an ability of assimilating pentose, and it is obtained by lowering activity of a gene involved in upstream of glyceraldehyde-3-phosphate in the Embden-Meyerhof pathway. The term “ability of assimilating pentose” used herein refers to an ability of assimilating pentose achieved by introducing an enzyme gene associated with pentose assimilation into a yeast strain that does not inherently have an ability of assimilating pentose (synonymous with “metabolizing ability”), and the term also refers to an inherent ability of assimilating pentose due to the presence of an enzyme gene associated with pentose assimilation. More specifically, the term “pentose” refers to aldopentose, such as ribose, arabinose, xylose, or lyxose, and ketopentose, such as ribulose or xylulose, although pentose is not particularly limited thereto. In some embodiments, the recombinant yeast strain of the present disclosure has an ability of assimilating xylose and/or arabinose among various types of pentoses. In more specific embodiments, the recombinant yeast strain has an ability of assimilating xylose.

Examples of yeast strains having the xylose-assimilating ability include a yeast strain that has acquired the xylose-assimilating ability via introduction of the xylose isomerase gene into a yeast strain that does not inherently have the xylose-assimilating ability and a yeast strain that has acquired the xylose-assimilating ability via introduction of another gene associated with xylose assimilation. Examples of yeast strains having the arabinose-assimilating ability include yeast strains that have each acquired the arabinose-assimilating ability via introduction of an L-arabinose isomerase gene, an L-ribulokinase gene, and an L-ribulose-5-phosphate-4-epimerase gene derived from prokaryotes and an L-arabitol-4-dehydrogenase gene and an L-xylose reductase gene derived from eukaryotes into a yeast strain that does not inherently have the arabinose-assimilating ability.

In the recombinant yeast strain of the present disclosure, an activity of a gene involved in upstream of glyceraldehyde-3-phosphate in the Embden-Meyerhof pathway is lowered.

The Embden-Meyerhof pathway is a pathway by which glucose is converted into pyruvate via glucose-6-phosphate, fructose-1,6-diphosphate, glyceraldehyde-3-phosphate, phosphoenolpyruvate and so on (Iwanami Seibutsugaku Jiten (Iwanami Dictionary of Biology), Vol. 4). The Embden-Meyerhof pathway comprises four reactions upstream of glyceraldehyde-3-phosphate: conversion of glucose into glucose-6-phosphate; conversion of glucose-6-phosphate into fructose-6-phosphate; conversion of fructose-6-phosphate into fructose-1,6-diphosphate; and conversion of fructose-1,6-diphosphate into glyceraldehyde-3-phosphate or dihydroxyacetone phosphate.

Lowering in an activity of a gene involved in upstream of glyceraldehyde-3-phosphate in the Embden-Meyerhof pathway is, in other words, lowering in an expression level of a gene encoding a relevant enzyme involved in each of the four reactions mentioned above, the presence of a substance that would inhibit activity of such enzyme, or substitution of a gene encoding such enzyme with a mutant gene encoding a mutant enzyme exhibiting low enzymatic activity. It should be noted that an embodiment in which the reaction is disrupted as a result of disruption of a gene encoding an enzyme associated with the reaction is not within the scope of the lowering in an activity of a gene involved in upstream of glyceraldehyde-3-phosphate in the Embden-Meyerhof pathway. When an activity of a gene involved in upstream of glyceraldehyde-3-phosphate in the Embden-Meyerhof pathway is lowered, specifically, a reaction of metabolizing glucose into glyceraldehyde-3-phosphate proceeds, but a glucose assimilation rate decreases. The glucose assimilation rate can be determined based on the glucose concentration in the medium.

In order to lower the expression levels of the genes encoding the enzymes involved in the above four reactions mentioned above, a promoter region in each gene may be substituted with a promoter exhibiting low promoter activity, or a terminator region in each gene may be substituted with a terminator region having effects of lowering the gene expression levels. Regarding a technique involving the use of a terminator region having effects of lowering the gene expression level, a reference may be made to U.S. Pat. No. 9,512,436 B2.

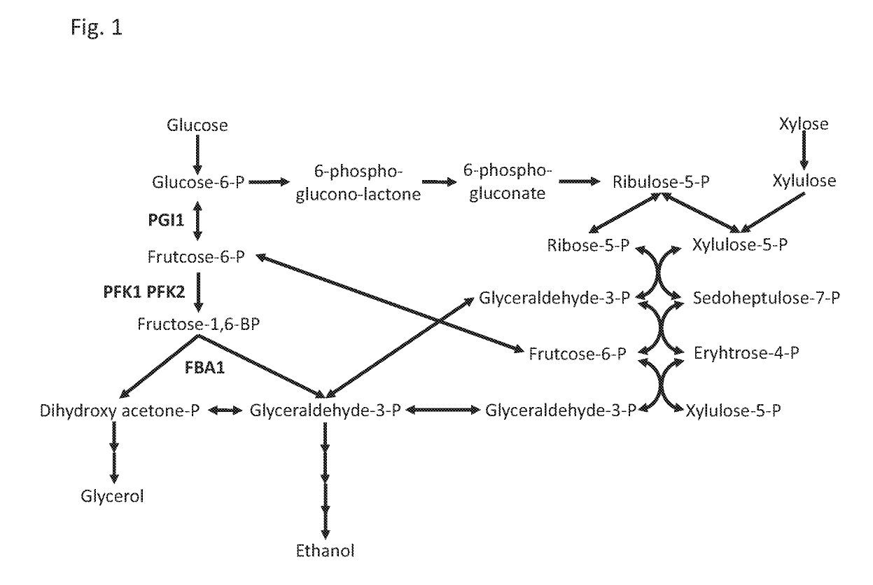

FIG. 1 schematically shows a process of ethanol fermentation from glucose and a process of ethanol fermentation from xylose including the four reactions. Conversion of glucose into glucose-6-phosphate involves hexokinase. Conversion of glucose-6-phosphate into fructose-6-phosphate involves glucose phosphate isomerase. Conversion of fructose-6-phosphate into fructose-1,6-diphosphate involves phosphofructokinase. Conversion of fructose-1,6-diphosphate into glyceraldehyde-3-phosphate or dihydroxyacetone phosphate involves fructose bisphosphate aldolase.

In some embodiments, accordingly, activity of at least one gene selected from the group consisting of the hexokinase gene, the glucose phosphate isomerase gene, the phosphofructokinase gene, and the fructose bisphosphate aldolase gene may be lowered in the recombinant yeast strain of the present disclosure. In more specific embodiments, activity of at least one gene selected from among the glucose phosphate isomerase gene. the phosphofructokinase gene, and the fructose bisphosphate aldolase gene may be lowered in the recombinant yeast strain of the present disclosure.

In the recombinant yeast strain of the present disclosure, a gene, activity of which is to be lowered, can be identified with the use of databases storing the nucleotide sequence of the gene, the amino acid sequence of the protein encoded by the gene of interest, and annotated information concerning functions of the protein. Examples of such databases include, but are not particularly limited to, DDBJ (DNA Data Bank of Japan), GenBank, EMBL, Unigene, TIGR Database, SGD (the Saccharomyces Genome Database), DBGET (GenomeNet). Entrez, SRS (EMBL), and KEGG.

With the use of the databases storing genetic information concerning Sccharomyces cerevisae of, for example, GenBank or SGD, a gene, activity of which is to be lowered, can be identified. Examples of genes encoding hexokinase include the HXK1 gene and the HXK2 gene of Sccharomyces cerevisae. An example of a gene encoding glucose phosphate isomerase is the PGI1 gene of Sccharomyces cerevisae. Examples of genes encoding phosphofructokinase include the PFK1 gene and the PFK2 gene of Sccharomyces cerevisae. The PFK1 gene encodes a subunit of phosphofructokinase that is different from the subunit encoded by the PFK2 gene. Since a heterooctamer is formed, phosphofructokinase activity would be lost in the absence of either one of the genes. An example of a gene encoding fructose bisphosphate aldolase is the FBA1 gene of Sccharomyces cerevisae.

A gene, activity of which is to be lowered, may be a paralogous gene or a homologous gene in the narrow sense having nucleotide and amino acid sequences different from those of the HXK1 gene, the HXK2 gene, the GLK1 gene, the PGI1 gene, the PFK1 gene, the PFK2 gene, or the FBA1 gene.

The recombinant yeast strain of the present disclosure may have an ability of assimilating, for example, xylose among various types of pentoses (i.e., the xylose-assimilating ability), specifically, it can assimilate xylose contained in a medium to generate ethanol. Xylose contained in a medium may be obtained by saccharification of xylan or hemicellulose comprising xylose as a constituent sugar. Alternatively, it may be supplied to a medium as a result of saccharification of xylan or hemicellulose contained in a medium by a saccharifying enzyme. The latter case refers to the so-called simultaneous saccharification and fermentation process.

Examples of yeast strains having xylose-metabolizing ability include a yeast strain that has acquired xylose-metabolizing ability as a result of introduction of a xylose isomerase gene into a yeast strain that does not inherently have xylose-metabolizing ability and a yeast strain that has acquired xylose-assimilating ability as a result of introduction of another xylose assimilation-associated gene.

The xylose isomerase gene (the XI gene) is not particularly limited, and a gene originating from any organism species may be used. For example, a plurality of the xylose isomerase genes derived from the intestinal protozoa of termites disclosed in JP 2011-147445 A can be used without particular limitation. Examples of the xylose isomerase genes that can be used include a gene derived from the anaerobic fungus Piromyces sp. strain E2 (JP 2005-514951 A), a gene derived from the anaerobic fungus Cyllamyces aberensis, a gene derived from a bacterial strain (i.e., Bacteroides thetalotaomicron), a gene derived from a bacterial strain (i.e., Clostridium phytofermentans), and a gene derived from the Streptomyces murinus cluster.

Specifically, a xylose isomerase gene derived from the intestinal protozoa of Reticulitermes speratus may be used. The nucleotide sequence of the coding region of the xylose isomerase gene derived from the intestinal protozoa of Reticulitermes speratus and the amino acid sequence of a protein encoded by such gene are as shown in SEQ ID NOs: 1 and 2, respectively.

The xylose isomerase gene is not limited to the gene identified by SEQ ID NO: 1 and SEQ ID NO: 2. It may be a paralogous gene or a homologous gene in the narrow sense having different nucleotide and amino acid sequences.

The xylose isomerase gene is not limited to the gene identified by SEQ ID NO: 1 and SEQ ID NO: 2. For example, it may be a gene comprising an amino acid sequence having 70% or higher, preferably 80% or higher, more preferably 90% or higher, and most preferably 95% or higher sequence similarity to or identity with the amino acid sequence as shown in SEQ ID NO: 2 and encoding a protein having xylose isomerase activity. The degree of sequence similarity or identity can be determined using the BLASTN or BLASTX Program equipped with the BLAST algorithm (at default settings). The degree of sequence similarity is determined by subjecting a pair of amino acid sequences to pairwise alignment analysis, identifying completely identical amino acid residues and amino acid residues exhibiting physicochemically similar functions, determining the total number of such amino acid residues, and calculating the percentage of all the amino acid residues subjected to comparison accounted for by the total number of such amino acid residues. The degree of sequence identity is determined by subjecting a pair of amino acid sequences to pairwise alignment analysis, identifying completely identical amino acid residues, and calculating the percentage of all the amino acid residues subjected to comparison accounted for by such amino acid residues.

Further, the xylose isomerase gene is not limited to the gene identified by SEQ ID NO: 1 and SEQ ID NO: 2. For example, it may be a gene comprising an amino acid sequence derived from the amino acid sequence as shown in SEQ ID NO: 2 by substitution, deletion, insertion, or addition of one or several amino acids and encoding a protein haying xylose isomerase activity. The term “several” used herein refers to, for example, 2 to 30, preferably 2 to 20, more preferably 2 to 10, and most preferably 2 to 5.

Furthermore, the xylose isomerase gene is not limited to the gene identified by SEQ ID NO: 1 and SEQ ID NO: 2. For example, it may be a gene hybridizing under stringent conditions to the full-length sequence or a partial sequence of a complementary strand of DNA comprising the nucleotide sequence as shown in SEQ ID NO: 1 and encoding a protein having xylose isomerase activity. Under “stringent conditions,” so-called specific hybrids are formed, but non-specific hybrids are not formed. For example, such conditions can be adequately determined with reference to Molecular Cloning: A Laboratory Manual (Third Edition). Specifically, the degree of stringency can be determined in accordance with the temperature and the salt concentration of a solution used for Southern hybridization and the temperature and the salt concentration of a solution used for the step of washing in Southern hybridization. Under stringent conditions, more specifically, the sodium concentration is 25 to 500 mM and preferably 25 to 300 mM, and the temperature is 42° C. to 68° C. and preferably 42° C. to 65° C. Further specifically, the sodium concentration is 5×SSC (83 mM NaCl, 83 mM sodium citrate), and the temperature is 42° C.

As described above, whether or not a gene comprising a nucleotide sequence that differs from the sequence as shown in SEQ ID NO: I or a gene encoding an amino acid sequence that differs from the sequence as shown in SEQ ID NO: 2 would function as a xylose isomerase gene may be determined by, for example, preparing an expression vector comprising the gene of interest incorporated into an adequate site between a promoter and a terminator, transforming an E. coil host using such expression vector, and assaying the xylose isomerase activity of the protein expressed. The term “xylose isomerase activity” refers to activity of isomerizing xylose into xylulose. Accordingly, xylose isomerase activity can be evaluated by preparing a xylose-containing solution as a substrate, allowing the target protein to react at an adequate temperature, and measuring the amount of xylose that has decreased and/or the amount of xylulose that has been generated.

In some embodiments, a gene comprising an amino acid sequence derived from the amino acid sequence as shown in SEQ ID NO: 2 by introduction of a particular mutation into a particular amino acid residue and encoding mutant xylose isomerase with improved xylose isomerase activity may be used as a xylose isomerase gene. A specific example of a gene encoding mutant xylose isomerase is a gene encoding an amino acid sequence derived from the amino acid sequence as shown in SEQ ID NO: 2 by substitution of asparagine with cysteine at position 337. Xylose isomerase activity of such mutant xylose isomerase is superior to that of wild-type xylose isomerase. In addition, mutant xylose isomerase is not limited to the xylose isomerase resulting from substitution of asparagine with cysteine at position 337. It may be xylose isomerase resulting from substitution of asparagine with an amino acid residue other than cysteine at position 337, xylose isomerase resulting from substitution of an amino acid residue at a position different from position 337, in addition to substitution of asparagine at position 337, or xylose isomerase resulting from substitution of an amino acid residue other than cysteine at position 337.

Meanwhile, examples of xylose metabolism-associated genes other than the xylose isomerase gene include a xylose reductase gene encoding a xylose reductase that converts xylose into xylitol, a xylitol dehydrogenase gene encoding a xylitol dehydrogenase that converts xylitol into xylulose, and a xylulokinase gene encoding a xylulokinase that phosphorylates xylulose to produce xylulose 5-phosphate. Xylulose 5-phosphate produced by a xylulokinase enters the pentose phosphate pathway, and it is then metabolized therein.

Examples of xylose metabolism-associated genes include, but are not particularly limited to, a xylose reductase gene and a xylitol dehydrogenase gene derived from Pichia stipitis and a xylulokinase gene derived from Saccharomyces cerevisiae (see Eliasson A. et al., Appl. Environ. Microbiol., 66: 3381-3386; and Toivari M. N. et al., Metab. Eng., 3: 236-249). In addition, xylose reductase genes derived from Candida tropicalis and Candida prapsilosis, xylitol dehydrogenase genes derived from Candida tropicalis and Candida prapsilosis, and a xylulokinase gene derived from Pichia stipitis can be used.

Examples of yeast strains that inherently have xylose-metabolizing ability include, but are not particularly limited to, Pichia stipitis, Candida tropicalis, and Candida prapsilosis.

The recombinant yeast strain of the present disclosure may have an ability of assimilating, for example, arabinose among various types of pentoses (i.e., the arabinose-assimilating activity), specifically, arabinose contained in a medium, and generating ethanol. The arabinose-assimilating activity can be imparted to a yeast strain that does not have arabinose-assimilating activity via introduction of the L-arabinose isomerase gene, the L-ribulokinase gene, or the L-ribulose-5-phosphate-4-epimerase gene derived from prokaryotes or the L-arabitol-4-dehydrogenase gene or L-xylose reductase gene derived from eukaryotes into such yeast strain.

The recombinant yeast strain of the present disclosure may further comprise other genes) introduced thereinto, and such other genes) are not particularly limited. For example, a gene involved in the sugar metabolism of glucose may be introduced into such recombinant yeast strain. For example, a recombinant yeast strain can have β-glucosidase activity resulting from the introduction of the β-glucosidase gene.

The term “β-glucosidase activity” used herein refers to the activity of catalyzing a hydrolysis reaction of a β-glycoside bond of a sugar. Specifically, β-glucosidase is capable of degrading a cellooligosaccharide, such as cellobiose, into glucose. The β-glucosidase gene can be introduced in the form of a cell-surface display gene. The term “cell-surface display gene” used herein refers to a gene that is modified to display a protein to be encoded by the gene on a cell surface. For example, a cell-surface display β-glucosidase gene results from fusion of a β-glucosidase gene with a cell-surface localized protein gene. A cell-surface localized protein is fixed and present on a yeast cell surface layer. Examples include agglutinative proteins, such as α- or a-agglutinin and FLO proteins. In general, a cell-surface localized protein comprises an N-terminal secretory signal sequence and a C-terminal GPI anchor attachment signal sequence. While a cell-surface localized protein shares properties with a secretory protein in terms of the presence of a secretory signal, its secretory signal differs in that the cell-surface localized protein is transported while fixed to a cell membrane through a GPI anchor. When a cell-surface localized protein passes through a cell membrane, a GPI anchor attachment signal sequence is selectively cut, it binds to a GPI anchor at a newly protruded C-terminal region, and it is then fixed to the cell membrane. Thereafter, the root of the GPI anchor is cut by phosphatidylinositol-dependent phospholipase C (PI-PLC). Subsequently, a protein separated from the cell membrane is integrated into a cell wall, fixed onto a cell surface layer, and then localized on a cell surface layer (see, for example, JP 2006-174767 A).

The β-glucosidase gene is not particularly limited, and an example is a β-glucosidase gene derived from Aspergillus aculeatus (Murai et al., Appl. Environ. Microbiol., 64: 4857-4861). In addition, a β-glucosidase gene derived from Aspergillus oryzae, a β-glucosidase gene derived from Clostridium cellulovorans, and a β-glucosidase gene derived from Saccharomycopsis fibligera may be used.

In addition to or other than the β-glucosidase gene, a gene encoding another cellulase-constituting enzyme may have been introduced into the recombinant yeast strain of the present disclosure. Examples of cellulase-constituting enzymes other than β-glucosidase include exo-cellobiohydrolases that liberate cellobiose from the terminus of crystalline cellulose (CBH1 and CBH2) and endo-glucanase (EG) that cannot degrade crystalline cellulose but cleaves a non-crystalline cellulose (amorphous cellulose) chain at random.

In particular, an example of another gene to be introduced into a recombinant yeast strain is a gene capable of promoting the use of xylose in a medium. A specific example thereof is a gene encoding xylulokinase having activity of generating xylulose-5-phosphate using xylulose as a substrate. The metabolic flux of the pentose phosphate pathway can be improved through the introduction of the xylulokinase gene.

Further, a gene encoding an enzyme selected from the group of enzymes constituting a non-oxidative process in the pentose phosphate pathway can be introduced into the recombinant yeast strain of the present disclosure. Examples of enzymes constituting a non-oxidative process in the pentose phosphate pathway include ribose-5-phosphate isomerase, ribulose-5-phosphate-3-epimerase, transketolase, and transaldolase. It is preferable that one or more genes encoding such enzymes be introduced, more preferable that two or more such genes be introduced in combination, further preferable that three or more genes be introduced in combination, and the most preferable that all of the genes above be introduced.

More specifically, the xylulokinase (XK) gene of any origin can be used without particular limitation. A wide variety of microorganisms, such as bacterial and yeast strains, Which assimilate xylulose, possess the XK gene. Information concerning XK genes can be obtained by searching the website of NCB1 or other institutions, according to need. Preferable examples of such genes include the XK genes derived from yeast strains, lactic acid bacteria, E. coli bacteria, and plants. An example of an XK gene is XKS1, which is an XK gene derived from the S. cerevisiae S288C strain (GenBank: 2729⁷9) (the nucleotide sequence and the amino acid sequence in the CDS coding region).

More specifically, a transaldolase (TAL) gene, a transketolase (TKL) gene, a ribulose-5-phosphate epimerase (RPE) gene, and a ribose-5-phosphate ketoisomerase (RKI) gene of any origin can be used without particular limitation. A wide variety of organisms comprising the pentose phosphate pathway possess such genes. For example, a common yeast strain such as S. cerevisiae possesses such genes. Information concerning such genes can be obtained from the website of NCBI or other institutions, according to need. Genes belonging to the same genus as the host eukaryotic cells, such as eukaryotic or yeast cells, are preferable, and genes originating from the same species as the host eukaryotic cells are more preferable. A TAL1 gene, a TKL1 gene and a TKL2 gene, an RPE1 gene, and an RKI1 gene can be preferably used as the TAL gene, the TKL genes, the RPE gene, and the RKI gene, respectively. Examples of such genes include a TAL1 gene derived from the S. cerevisiae S288 strain (GenBank: U19102), a TKL1 gene derived from the S. cerevisiae S288 strain (GenBank: X73224), an RPE1 gene derived from the S. cerevisiae S288 strain (GenBank: X83571), and an RKI1 gene derived from the S. cerevisiae S288 strain (GenBank: Z75003).

### <Production of Recombinant Yeast Strain>

The recombinant yeast strain of the present disclosure can be produced by, for example, introducing the xylose metabolism-associated gene or the arabinose metabolism-associated gene into a yeast strain having no ability of metabolizing pentose, such as xylose or arabinose, and modifying the yeast strain to lower activity of a gene involved in upstream of glyceraldehyde-3-phosphate in the Embden-Meyerhof pathway. Alternatively, the recombinant yeast strain of the present disclosure can be produced by modifying, for example, the yeast strain having the pentose metabolizing ability to lower activity of the gene involved in upstream of glyceraldehyde-3-phosphate in the Embden-Meyerhof pathway. It should be noted that another gene described above may be introduced when producing the recombinant yeast strain of the present disclosure.

In order to lower the gene expression level, a mutation that lowers the transcription level may be introduced into an endogenous promoter of the gene, the endogenous promoter may be substituted with a promoter of another gene exhibiting a low expression level, or either of a pair of genes present in the diploid recombinant yeast strain may be deleted. Examples of techniques for suppressing gene expression include the transposon technique, the transgene technique, post-transcriptional gene silencing, the RNAi technique, the nonsense mediated decay (NMD) technique, the ribozyme technique, the anti-sense technique, the miRNA (micro-RNA) technique, and the siRNA (small interfering RNA) technique. A terminator region having effects of lowering the gene expression level may be used (U.S. Pat. No. 9,512,436 B2).

When the xylose metabolism-associated gene, the arabinose metabolism-associated gene, and other genes are to be introduced into a yeast strain, such genes may be simultaneously introduced thereinto, or such genes may be successively introduced with the use of different expression vectors.

Examples of host yeast strains that can be used include, but are not particularly limited to, Candida Shehatae, Pichia stipitis, Pachysolen tannophilus, Saccharomyces cerevisiae, and Schizosaccaromyces pombe, with Saccharomyces cerevisiae being particularly preferable. Experimental yeast strains may also be used from the viewpoint of experimental convenience, or industrial (practical) strains may also be used from the viewpoint of practical usefulness. Examples of industrial strains include yeast strains used for the production of wine, sake, and shochu.

Use of a host yeast strain having homothallic properties is preferable. According to the technique disclosed in JP 2009-34036 A, multiple copies of genes can be easily introduced into a genome with the use of a yeast strain having homothallic properties. The term “yeast strain having homothallic properties” has the same meaning as the term “homothallic yeast strain.” Yeast strains having homothallic properties are not particularly limited, and any yeast strains can be used. An example of a yeast strain having homothallic properties is, but is not limited to, the Saccharomyces cerevisiae OC-2 train (NBRC2260). Examples of other yeast strains having homothallic properties include an alcohol-producing yeast (Taiken No. 396, NBRC0216) (reference: “Alcohol kobo no shotokusei” (“Various properties of alcohol-producing yeast”), Shuken Kaiho, No. 37, pp. 18-22, 1998.8), an ethanol-producing yeast isolated in Brazil and in Japan (reference: “Brazil to Okinawa de bunri shita Saccharomyces cerevisiae yaseikabu no idengakuteki seishitsu” (“Genetic properties of wild-type Saccharomyces cerevisiae isolated in Brazil and in Okinawa”), the Journal of the Japan Society for Bioscience, Biotechnology, and Agrochemistry, Vol. 65, No. 4, pp. 759-762, 1991.4), and 180 (reference: “Alcohol Hakkoryoku no tsuyoi kobo no screening” (“Screening of yeast having potent alcohol-fermenting ability”), the Journal of the Brewing Society of Japan, Vol. 82, No. 6, pp. 439-443, 1987.6). In addition, the HO gene may be introduced into a yeast strain exhibiting heterothallic phenotypes in an expressible manner, and the resulting strain can be used as a yeast strain having homothallic properties. That is, the term “yeast strain having homothallic properties” used herein also refers to a yeast strain into which the HO gene has been introduced in an expressible manner.

Promoters of genes to be introduced are not particularly limited. For example, promoters of glyceraldehyde-3-phosphate dehydrogenase gene (TDH3), the 3-phosphoglycerate kinase gene (PGK1), and the high-osmotic pressure response 7 gene (HOR7) can be used. The promoter of the pyruvate decarboxylase gene (PDC1) is particularly preferable in terms of its high capacity for expressing target genes in a downstream region at high levels.

Specifically, such gene may be introduced into the yeast genome together with an expression-regulated promoter or another expression-regulated region. Such gene may be introduced into a host yeast genome in such a manner that expression thereof is regulated by a promoter or another expression-regulated region of a gene that is inherently present therein.

The gene can be introduced into the genome by any conventional technique known as a yeast transformation technique. Specific examples include, but are not limited to, electroporation (Meth. Enzym., 194, p. 182, 1990), the spheroplast technique (Proc. Natl. Acad. Sci., U.S.A., 75, p. 1929, 1978), and the lithium acetate method (J. Bacteriology, 153, p. 163, 1983; Proc. Natl. Acad. Sci., U.S.A., 75, p. 1929, 1978; Methods in yeast genetics, 2000 Edition: A Cold Spring Harbor Laboratory Course Manual).

### <Production of Ethanol>

When producing ethanol with the use of the recombinant yeast strain described above, ethanol fermentation is carried out by culture in a medium containing at least pentose, such as xylose or arabinose. Specifically, a medium in which ethanol fermentation is carried out contains, as a carbon source, at least metabolizable pentose. The medium may be supplemented with another carbon source, such as glucose, in advance.

A pentose, such as xylose or arabinose, that is contained in a medium to be used for ethanol fermentation can be derived from a biomass. In other words, a medium to be used for ethanol fermentation may comprise a cellulosic biomass and hemicellulase that generates pentose, such as xylose or arabinose, through saccharification of hemicellulose contained in a cellulosic biomass. The cellulosic biomass may have been subjected to a conventional pretreatment technique. Examples of pretreatment techniques include, but are not particularly limited to, degradation of a lignin with a microorganism and grinding of a cellulosic biomass. For example, a ground cellulosic biomass may be subjected to pretreatment, such as soaking thereof in a dilute sulfuric acid solution, alkaline solution, or ionic solution, hydrothermal treatment, or fine grinding. Thus, the efficiency of biomass saccharification can be improved.

When producing ethanol with the use of the recombinant yeast strain described above, the medium may further comprise cellulose and cellulase. In such a case, the medium contains glucose generated by the action of cellulase imposed upon cellulose. When a medium used for ethanol fermentation contains cellulose, such cellulose can be derived from a biomass. In other words, a medium used for ethanol fermentation may comprise cellulase that is capable of saccharifying cellulase contained in a cellulosic biomass.

A saccharified solution resulting from saccharification of a cellulosic biomass may be added to the medium used for ethanol fermentation. In such a case, the saccharified solution contains remaining cellulose or cellulose and pentose, such as xylose or arabinose, derived from hemicellulose contained in a cellulosic biomass.

As described above, the method for producing ethanol of the present disclosure comprises a step of ethanol fermentation involving the use of at least pentose, such as xylose or arabinose, as a saccharide source. According to the method for producing ethanol of the present disclosure, ethanol can be produced through ethanol fermentation using pentose, such as xylose or arabinose, as a saccharide source. According to the method for producing ethanol with the use of the recombinant yeast strain of the present disclosure, ethanol fermentation is followed by recovery of ethanol from the medium. Ethanol may be recovered by any conventional means without particular limitation. After the completion of the process of ethanol fermentation mentioned above, for example, a liquid layer containing ethanol is separated from a solid layer containing the recombinant yeast strain or solid matter via solid-solution separation. Thereafter, ethanol contained in a liquid layer is separated and purified by distillation, so that highly purified ethanol can be recovered. The degree of ethanol purification can be adequately determined in accordance with the purpose of use of the ethanol.

In general, glycerin is known as a representative by-product of ethanol production via fermentation using a yeast strain. In order to improve an ethanol yield in ethanol production via fermentation, it is important to reduce the amount of glycerin produced. When genes associated with the glycerin production pathway from glyceraldehyde-3-phosphate GPD1, GPD2, GPP1, and GPP2 genes) are disrupted or expression levels thereof are lowered to reduce the amount of glycerin produced, however, drawbacks, such as a lowered ethanol production rate, are pointed out (Appl. Environ. Microbiol., 2011, 77, 5857-5867; Appl. Environ. Microbiol., 2013, 79, 3273-3281).

As described in the examples below, the recombinant yeast strain of the present disclosure is characterized by a very low glycerin production level. In particular, the recombinant yeast strain of the present disclosure is advantageous in terms of a low glycerin production level even if the gene associated with the glycerin production pathway from glyceraldehyde-3-phosphate is not disrupted or an expression level thereof is not lowered. With the use of the recombinant yeast strain of the present disclosure, accordingly, an ethanol yield superior to that of a yeast strain in which the gene associated with the glycerin production pathway from glyceraldehyde-3-phosphate is disrupted or an expression level thereof is lowered can be achieved.

The method for producing ethanol of the present disclosure may employ the so-called simultaneous saccharification and fermentation process in which the step of saccharification of cellulose contained in a medium with a cellulase proceeds simultaneously with the step of ethanol fermentation involving the use of saccharide sources (i.e., pentose, such as xylose or arabinose, and glucose generated by saccharification). With the simultaneous saccharification and fermentation process, the step of saccharification of a cellulosic biomass is carried out simultaneously with the process of ethanol fermentation.

Methods of saccharification are not particularly limited. For example, an enzymatic method involving the use of a cellulose preparation, such as cellulose or hemicellulase, may be employed. A cellulase preparation contains a plurality of enzymes involved in degradation of a cellulose chain and a hemicellulose chain, and it exhibits a plurality of types of activity, such as endoglucanase activity, endoxylanase activity, cellobiohydrolase activity, glucosidase activity, and xylosidase activity. Cellulase preparations are not particularly limited, and examples include cellulases produced by Trichoderma reesei and Acremonium cellulolyticus. Commercially available cellulase preparations may also be used.

In the simultaneous saccharification and fermentation process, a cellulase preparation and the recombinant microorganism are added to a medium containing a cellulosic biomass (a biomass after pretreatment may be used), and the recombinant yeast strain is cultured at a given temperature. Culture may be carried out at any temperature without particular limitation, and the temperature may be 25° C. to 45° C., and preferably 30° C. to 40° C. from the viewpoint of ethanol fermentation efficiency. The pH level of the culture solution is preferably 4 to 6. Agitation or shake culture may be employed. Alternatively, the simultaneous saccharification and fermentation process may be carried out irregularly in such a manner that saccharification is first carried out at an optimal temperature for an enzyme (40° C. to 70° C.), temperature is lowered to a given level (30° C. to 40° C.), and a yeast strain is then added thereto.

### EXAMPLES

Hereafter, the present disclosure is described in greater detail with reference to the examples, although the technical scope of the present disclosure is not limited to these examples.

### Example 1

In the present example, a recombinant yeast strain having the xylose-metabolizing ability was prepared by lowering activity of a gene (the PGI1 gene) encoding glucose phosphate isomerase, which is a gene involved in upstream of glyceraldehyde-3-phosphate in the Embden-Meyerhof pathway.

### <Production of Vectors for Gene Introduction>

### (1) Plasmid for X1, XKS1, TKL1, TAL1, RKI1, and RPE1 Gene Introduction and for GRE3 Gene Disruption

A plasmid (pUC-5U_GRE3-P_HOR7-TKL1-TAL1-FBA1_P-P_ADH1-RPE1-RKI1-TEF1_P-P_TDH1-XI_N337C-T_DIT1-P_TDH3-XKS1-T_HIS3-LoxP-G418-LoxP-3U_GRE3) was prepared. This plasmid comprises, at the GRE3 gene locus, a sequence necessary for disruption of the GRE3 gene and introduction of the following genes into yeast: a mutant gene with an improved xylose assimilation rate resulting from substitution of asparagine with threonine at amino acid 377 of the xylose isomerase gene derived from the intestinal protozoa of Reticulitermes speratus (see XI_N377C; WO 2014/156194), a yeast-derived xylulokinase (XKS1) gene, the transketolase 1 (TKL1) gene, the transaldolase 1 (TAL1) gene, the ribulose-phosphate-epimerase 1 (RPE1) gene, and the ribose-phosphate ketoisomerase (RKI1) gene of the pentose phosphate pathway.

This plasmid was constructed to comprise: the TKL1 gene derived from the Saccharomyces cerevisiae BY4742 strain in which an HOR7 promoter is added on the 5′ side; the TAL1 gene in which an FBA1 promoter is added; the RKI1 gene in which an ADH1 promoter is added; the RPE1 gene in which a TEF1 promoter is added; XI_N337C in which a TDH1 promoter and a DIT1 terminator are added (prepared through the total synthesis on the basis of a sequence designed by changing codons over the entire region in accordance with the frequency of codon usage of the yeast strain); the XKS1 gene in which a TDH3 promoter and an HIS3 terminator are added; a gene sequence (5U_GRE3) comprising an upstream region of approximately 700 bp from the 5′ terminus of the GRE3 gene and a DNA sequence (3U_GRE3) comprising a downstream region of approximately 800 by from the 3′ terminus of the GRE3 gene, which are regions to be integrated into the yeast genome via homologous recombination; and a gene sequence (a G418 marker) comprising the G418 gene, which is a marker. The LoxP sequences were introduced on the both sides of the marker gene, so that the marker can be removed.

In addition, each DNA sequence can be amplified via PCR using the primers listed in Table 1 below. In order to ligate DNA fragments, a desired plasmid to be obtained as a final product was prepared in the following manner. A DNA sequence was added to each primer, so that the DNA sequence would overlap with its adjacent DNA sequence by approximately 15 bp. The primers were used to amplify desired DNA fragments using, as templates, the Saccharomyces cerevisiae BY4742 genome, DNA of the XI_N337C-synthesizing gene, and synthetic DNA of the LoxP sequence. The DNA fragments were sequentially ligated using an In-Fusion HD Cloning Kit (Takara Bio Inc.) or the like and then cloned into the pUC19 plasmid.

### (2) Plasmid for Conversion of PGI1 Gene Terminator

A plasmid (pUC-5U_PGI1-PGI1-T_GIC1-LoxP-P_CYC1-G418-T_URA3-LoxP-3U_PGI1) was prepared. This plasmid comprises a sequence necessary for substitution of the PGI1 gene terminator with a terminator exhibiting a low expression level (i.e., GIC1 (US20130244243)). This plasmid was constructed to comprise a DNA sequence (5U_PGI1) comprising an upstream region of approximately 800 by of the PGI1 gene, ORF of PGI1, a DNA sequence (3U_PGI1) comprising a downstream region of approximately 750 by of the PGI1 gene, which are regions to be integrated into the yeast genome via homologous recombination and substituted with a terminator of the phosphoglucose isomerase gene (PGI1) and a gene sequence (a G418 marker) comprising the G418 gene, which is a marker.

Each DNA sequence can be amplified via PCR using the primers listed in Table 1 below. In order to ligate DNA fragments, a desired plasmid to be obtained as a final product was prepared in the following manner. A DNA sequence was added to each primer, so that the DNA sequence would overlap with its adjacent DNA sequence by approximately 15 bp. The primers were used to amplify desired DNA fragments using, as templates, a plasmid (pUC-5U_GRE3-P_HOR7-TKL1-TAL1-FBA1_P-P_ADH1-RPE1-RKI1-TEF1_P-P_TDH1- XI_N337C-T_DIT1-P_TDH3-XKS1-T_HIS3-LoxP-G418-LoxP-3U_GRE3) or genomic DNA of the yeast OC2 strain. The DNA fragments were sequentially ligated using an In-Fusion HD Cloning Kit or the like and then cloned into the pUC19 plasmid.

TABLE 1

| | | |
|---|---|---|
| | | SEQ ID |
| Amplified DNA fragment | Primer sequence (5′-3′) | No: |

pUC-5U_GRE3-P_HOR7-TKL1-TAL1-FBA1_P-P_ADH1-RPE1-RKI1-TEF1_P-P_TDH1-XI_N337C-T_DIT1-
P_TDH3-XKS1-T_HIS3-LoxP-G418-LoxP-3U_GRE3

| | | |
|---|---|---|
| 5U_GRE3 | TGGGAATATTACCGCTCGAAG | 3 |
| | CTTTAAAAAATTTCCAATTTTCCTTTACG | 4 |
| HOR7 promoter | GGAAATTTTTTAAAGTCGCAGCCACGGGTCAAC | 5 |
| | GTGAATTGAGTCATTTTTTATTATTAGTCTTTTTTTTTTTTGACAATATC | 6 |
| TKL1 | ATGACTCAATTCACTGACATTGATAAGCTAG | 7 |
| (terminator region | CCTTAAATCAACGTCATATTCTTTATTGGCTITATAC | 8 |
| included) | | |
| TALI1 | GACGTTGATTTAAGGTGGTTCCGG | 9 |
| (terminator region | ATGTCTGAACCAGCTCAAAAGAAAC | 10 |
| included) | | |
| FBA1 promoter | AGCTGGTTCAGACATTTTGAATATGTATTACTTGGTTATGGTTATATATGAC | 11 |
| | ACTGGTAGAGAGCGACTTTGTATGC | 12 |
| ADH1 promoter | CAAAGTCGCTCTCTACCAGTCGCTTTCAATTCATTTGGGTG | 13 |
| | TGTATATGAGATAGTTGATTGTATGC | 14 |
| RPE1 | ACTATCTCATATACAATGGTCAAACCAATTATAGCTCCC | 15 |
| (terminator region | AAATGGATATTGATCTAGATGGCGG | 16 |
| included) | | |
| RKI1 | GATCAATATCCATTTCTTGGTGTGTCATCGGTAGTAACGCC | 17 |
| (terminator region | AGTTTTAATTACAAAATGGCTGCCGGTGTCCCAAA | 18 |
| included) | | |
| TEF1 promoter | TTGTAATTAAAACTTAGATTAGATTGCTATGCTTTC | 19 |
| | AGGAACAGCCGTCAAGGG | 20 |
| TDH1 promoter | TTGACGGCTGTTCCTCTTCCCTTTTACAGTGCTTC | 21 |
| | TTTGTTTTGTGTGTAAATTTAGTGAAGTACTG | 22 |
| XI_N337C | TACACACAAAACAAAATGTCTCAAATTTTTAAGGATATCCC | 23 |
| | AGCGCTCTTACTTTAGCGATCGCACTAGTTTATTGAAAC | 24 |
| DIT1 terminator | TAAAGTAAGAGCGCTACATTGGTCTACC | 25 |
| | TAACATTCAACGCTATTACTCCGCAACGCTTTTCTG | 26 |
| TDH3 promoter | TAGCGTTGAATGTTAGCGTCAACAAC | 27 |
| | TTTGTTTGTTTATGTGTGTTTATTCGAAACTAAGTTCTTGG | 28 |
| XKS1 | ACATAAACAAACAAAATGTTGTGTTCAGTAATTCAGAGACAG | 29 |
| | AAATAATCGGTGTCATTAGATGAGAGTCTTTTCCAGTTC | 30 |
| HIS3 terminator | TGACACCGATTATTTAAAGCTGCAG | 31 |
| | AGAGCGCGCCTCGTTCAG | 32 |
| LoxP | AACGAGGCGCGCTCTAATTCCGCTGTATAGCTC | 33 |
| (linker sequence | ATAATGTATGCTATACGAAGTTATAGGGAAAGATATGAGCTATAC | 34 |
| included) | | |
| CYC1 promoter | TATAGCATACATTATACGAAGTTATACGACATCGTCGAATATG | 35 |
| | TATTAATTTAGTGTGTGTATTTGTGTTTGTGTG | 36 |
| G418 | CACACTAAATTAATAATGAGCCATATTCAACGGG | 37 |
| | TTTAGTAGACATGCATTACAACCAATTAACCAATTCTG | 38 |
| URA3 terminator | TGCATGTCTACTAAACTCACAAATTAGAGCTTCAATT | 39 |
| | ATAATGTATGCTATACGAAGTTATGGGTAATAACTGATATAATTAAATTGAAGC | 40 |
| LoxP | TATAGCATACATTATACGAAGTTATTGACACCGATTATTTAAAGCTG | 41 |
| (linker sequence | ATTTTACTGGCTGGAGTATGCTGCAGCTTTAAATAATCG | 42 |
| included) | | |
| 3U_GRE3 | TCCAGCCAGTAAAATCCATACTCAAC | 43 |
| | GTCTTTTTGCCAGCCAGTCC | 44 |
| pUC19 | CACACCTTCCCCCTTGATCCTCTAGAGTCGACC | 45 |
| | GCGGTAATATTCCCAGATCCCCGGGTACCGAGCTC | 46 |

pUC-5U_RGI1-PGI1-T_GIC1-LoxP-P_CYC1-G418-T_URA3-LoxP-3U_PGI1

| | | |
|---|---|---|
| 5U_PGI1 and PGI1 | TAACATGCGGCATTTCCTGG | 47 |
| | TCACATCCATTCCTTGAATTGATTG | 48 |
| GIC1 terminator | AAGGAATGGATGTGAACTAGTTTTCTTCTTTCCTCCTCTTC | 49 |
| | CGTTGGTTGAAACGTTGTCTG | 50 |
| G418 marker | ACGTTTCAACCAACGAATTCCGCTGTATAGCTCATATC | 51 |
| | TTAAGAGCGATTTGTGTATGCTGCAGCTTTAAATAATCG | 52 |
| PGI1 | ACAAATCGCTCTTAAATATATACCTAAAGAAC | 53 |
| | ATCTTGCCCTATTGCATTCCC | 54 |
| pUC19 | GCAATGGATACTTCTGGGGATCCTCTAGAGTCG | 55 |
| | AAATGCCGCATGTTAGGGTACCGAGCTCGAATTCACTGGCCGTCG | 56 |

### <Production of Yeast Strains Comprising Vectors Introduced Thereinto>

The diploid yeast strains, Saccharomyces cerevisiae OC2-T (NBRC2260), were designated as host strains. Yeast strains were transformed using the Frozen-EZ Yeast Transformation II (ZYMO RESEARCH) in accordance with the protocols included therein.

A homologous recombination region of the plasmid prepared in (1) above (pUC-5U_GRE3-P_HOR7-TKL1-TAL1-FBA1_P-P_ADH1-RPE1-RKI1-TEF1_P-P_TDI1-XI_N337C-T_DIT1-P_TDH3-XKS1-T_HIS3-LoxP-G418-LoxP-3U_GRE3) was amplified via PCR, the OC2U strains were transformed using the resulting fragment, the resulting transformants were applied to a G418-containing YPD agar medium, and the grown colonies were then subjected to acclimatization. The acclimatized elite strain was designated as the Uz2126 strain. This strain was applied to a sporulation medium (1% potassium phosphate, 0.1% yeast extract, 0.05% glucose, and 2% agar) for sporulation, and a diploid of the strain was formed by utilizing homothallism. The strain in which the mutant XI, TKL1, TAL1, RPE1, RKI1 and XKS1 genes had been incorporated into the GRE3 gene locus of a diploid chromosome and the GRE3 gene had been disrupted was obtained. The resulting strain was designated as the Uz2126-2-1dS strain.

Subsequently, fragments amplified via PCR from the homologous recombination regions of the plasmid prepared in (2) above (pUC-5U_PGI1-PGI1-T_GIC1-LoxP-P_CYC1-G418-T_URA3-LoxP-3U_PGI1) were used to transform the Uz2126-2-1dS strain, the resulting strains were applied to a G418-containing YPD agar medium, and the grown colonies were subjected to acclimatization. Heterozygous recombination (1 copy) was observed in the above acclimatized elite strain and designated as the Uz2154-2-6 strain.

Sporulation was induced in a sporulation medium for the Uz2154-2-6 strain, a diploid thereof was formed utilizing homothallism, and the resulting strain was designated as the Uz2154-2-6dS strain. The genotypes of the strains produced as the final products are summarized as follows.

TABLE 2

| Strain | Genotype |
|---|---|
| Uz2126-2-1dS | gre3:: XI_N337C XKS1 TKL1 TAL1 RKI1 RPE1 |
| Uz2154-2-6dS | PGI1::PGI1-GIC1t G418 gre3:: XI_N337C XKS1 |
| | TKL1 TAL1 RKI1 RPE1 |

### <Fermentation Test>

From among the strains obtained above, 5 strains each were selected and subjected to a fermentation test in flasks. The test strains were inoculated into 100-ml baffled flasks each comprising 20 ml of YPD liquid medium (yeast extract concentration: 10 g/l; peptone concentration: 20 g/l; and glucose concentration: 20 g/l), and culture was conducted at 30° C. and 120 rpm for 24 hours. The strains were harvested and inoculated into 10-ml flasks each comprising 8 ml of a medium for ethanol production of a different composition (cell concentration: 0.3 g dry cells/l), and the fermentation test was carried out via agitation culture (80 rpm; an amplitude: 35 mm) while adjusting a temperature at 31° C. or 34° C. A rubber stopper into which a needle (i.d.: 1.5 mm) has been inserted was used to cap each flask, and a check valve was mounted on the tip of the needle to maintain the anaerobic conditions in the flask.

Glucose, xylose, and ethanol in the fermentation liquor were assayed via HPLC (LC-10A; Shimadzu Corporation) under the conditions described below.

### Column: Aminex HPX-87H

Mobile phase: 0.01N H₂SO₄
Flow rate: 0.6 ml/min

### Temperature: 30° C.

Detection apparatus: Differential refractometer (RID-10A)

### <Results of Fermentation Test 1>

The results of the fermentation test are shown in Table 3. The results shown in Table 3 were obtained by the test performed with the use of a medium containing glucose (50 g/l), xylose (100 g/l), yeast extract (10 g/l), peptone (20 g/l), and acetic acid (1.68 g/l) at a fermentation temperature of 31° C. Concentration of the substances, glycerin yield, and ethanol yield are average values of the results concerning 5 recombinant strains independently obtained 66 hours after the initiation of fermentation.

TABLE 3

| | | | | | | Glycerin yield | Ethanol yield |
|---|---|---|---|---|---|---|---|
| | | | | | Acetic | relative to | relative to |
| | Ethanol | Xylose | Glucose | Glycerin | acid | consumed | consumed |
| | (g/L) | (g/L) | (g/L) | (g/L) | (g/L) | saccharide | saccharide |
| Uz2126-2-1dS | 45.1 | 31.2 | 0.0 | 1.96 | 1.96 | 0.0181 | 0.832 |
| control | | | | | | | |
| Uz2154-2-6dS | 50.3 | 27.0 | 0.0 | 1.13 | 1.84 | 0.0100 | 0.892 |
| PGI1::PGI1-GIC1t | | | | | | | |

As shown in Table 3, the xylose assimilation rate was increased in a strain in which PGI1 gene activity was lowered, glycerin yield was significantly lowered, and ethanol yield was significantly improved, compared with a control strain. In addition, acetic acid assimilation capacity of the strain in which PGI1 gene activity was lowered was found to be superior to that of the control strain.

### <Growth Test>

With the use of the strains obtained above and the control strains, a growth test was carried out using a medium containing glucose as a carbon source. Culture was conducted in YPD liquid medium (yeast extract concentration: 10 g/l; peptone concentration: 20 g/l; and glucose concentration: 20 g/l) for 12 hours, and the strains were harvested and inoculated into 50-ml culture tubes (TPP) each containing 10 ml of YPD liquid medium to perform the growth test. The test was carried out using the real-time cell culture monitoring apparatus (RTS-1, BIOSAN LTD.) While conducting culture at 30° C., and OD of the culture solution was measured (850 nm) at intervals of 10 minutes. Table 4 shows the results of measurements of specific growth rate of each strain.

TABLE 4

| | Uz2126-2-1dS | Uz2154-2-6dS |
|---|---|---|
| Maximum specific growth rate μ(h⁻¹) | 1.21 | 0.904 |

As shown in Table 4, the specific growth rate of the strain with lowered activity of the PGI1 gene was found to be low in a medium containing glucose as a carbon source, compared with that of the control strain. As demonstrated in this example, lowered activity of a gene involved in upstream of glyceraldehyde-3-phosphate in the Embden-Meyerhof pathway, such as a glucose phosphate isomerase gene, would lead to a lowered glucose assimilation rate. Concerning xylose assimilation, in contrast, a gene involved in upstream of glyceraldehyde-3-phosphate in the Embden-Meyerhof pathway is not a direct metabolite system. Thus, it is considered less influential on xylose assimilation than on glucose assimilation.

In general, the glucose assimilation rate is significantly higher than the xylose assimilation rate in a strain comprising the xylose-assimilating pathway introduced thereinto, and glucose assimilation is often followed by xylose assimilation. In the strain with lowered activity of the PGI1 gene activity obtained in the present example, a glucose assimilation rate can be decreased to some extent. In the glycolytic pathway (see FIG. 1) located downstream of the glucose phosphate isomerase where the glucose metabolizing pathway joins the xylose metabolizing pathway, it was considered that assimilation of xylose-derived carbon sources would be relatively preferential, and ethanol productivity would be improved.

1. A recombinant yeast strain having an ability of assimilating pentose, which is obtained by lowering activity of a gene involved in upstream of glyceraldehyde-3-phosphate in the Embden-Meyerhof pathway.

2. The recombinant yeast strain according to claim 1, wherein the gene involved in upstream of glyceraldehyde-3-phosphate in the Embden-Meyerhof pathway is at least one gene selected from the group consisting of the hexokinase gene, the glucose phosphate isomerase gene, the phosphofructokinase gene, and the fructose bisphosphate aldolase gene.

3. The recombinant yeast strain according to claim 1, wherein the amount of glycerin production is reduced, compared with a yeast strain that retains the gene activity.

4. The recombinant yeast strain according to claim 1, wherein the pentose is xylose and/or arabinose.

5. The recombinant yeast strain according to claim 1, which comprises the xylose isomerase gene introduced thereinto and has the xylose-assimilating ability.

6. The recombinant yeast strain according to claim 5, which further comprises a xylulokinase gene introduced thereinto.

7. The recombinant yeast strain according to claim 1, which comprises a gene encoding an enzyme selected from a group of enzymes constituting a non-oxidative process in the pentose phosphate pathway introduced thereinto.

8. The recombinant yeast strain according to claim 7, wherein the group of enzymes constituting a non-oxidative process in the pentose phosphate pathway includes ribose-5-phosphate isomerase, ribulose-5-phosphate-3-epimerase, transketolase, and transaldolase.

9. A method for producing ethanol comprising a step of ethanol fermentation by culturing the recombinant yeast strain according to claim 1 in a medium containing assimilable pentose.

10. The method for producing ethanol according to claim 9, wherein the medium contains cellulose and the ethanol fermentation proceeds simultaneously at least with cellulose saccharification.


SEQUENCE LISTING (56 sequences)

SEQ ID NO: 1 - DNA, 1320 bp, Reticulitermes speratus
  CDS (1)..(1320)
atgtctcaaatttttaaggatatcccagttattaaatatgaaggtccagcttccaagaatcctttgagtttcaaatactacgatgcaaacaaggttattgatggtaaaccaatgaaggaacatttgagatacgcaatggcttggtggcataatttgtgtgctaccggtcaagatatgtttggtcctggtactgcagataaatccttcggtagtaagacagttggtaccatggaacatgcacatgctaaagttgatgctggttttgaattcatgtccaagttgggtgttgaatacttctgtttccatgatgctgatttggttccagaagcagatactttgagtgaaacaaacaaaagattggatgaaatcgctgaacatatcgttgctaagcaaaaggcaactggtattaaatgtttgtggggtacagcaaatttgttttctaaccctagattcttaaatggttctggttcttcaaactcagctgatgtttatgcatacgctgcagctcaaattaaaaaggctttggatttgactgttaaatttggtggtgttggttatgttttctggggtggtagagaaggttacgaaaccttgttgaacactgatgttaagttcgaacaagaaaacatcgctaacttgatgcatttggcagttacttacggtagatcaatcggttttaaaggtgacttctacattgaaccaaaacctaaggaaccaacaaagcatcaatatgattttgatgcagctactacaattggtttcattagacaatacggtttggaaaaggatttcaagttgaacatcgaagcaaaccatgctacattagcaggtcataccttccaacatgatttgagaatctctgctattaatggcatgttaggttcagttgatgcaaacacaggtgacccattgttaggttgggataccgatgaatttccttattccgtttacgataccactttggctatgtacgaaattattaaggcaggtggtttgaccggtggtttgaattttgattccaaggttagaagaccaagttacacacatgaagatttgttttacggtttcattttgggtatggattctttcgctttgggtttgattaaagcaaaggctttgattgcagatggtagattggattcattcgttaaggatagatacgcttcttacggttcaggtattggtgctaagattagagatcattctgcaactttggaagaattagcagcttatgcattagctaaagatacagttgctttgcctggttccggtagacaagaatacttagaaagtattattaaccaaattttgtttcaataa

SEQ ID NO: 2 - protein, 439 aa, Reticulitermes speratus
MSQIFKDIPVIKYEGPASKNPLSFKYYDANKVIDGKPMKEHLRYAMAWWHNLCATGQDMFGPGTADKSFGSKTVGTMEHAHAKVDAGFEFMSKLGVEYFCFHDADLVPEADTLSETNKRLDEIAEHIVAKQKATGIKCLWGTANLFSNPRFLNGSGSSNSADVYAYAAAQIKKALDLTVKFGGVGYVFWGGREGYETLLNTDVKFEQENIANLMHLAVTYGRSIGFKGDFYIEPKPKEPTKHQYDFDAATTIGFIRQYGLEKDFKLNIEANHATLAGHTFQHDLRISAINGMLGSVDANTGDPLLGWDTDEFPYSVYDTTLAMYEIIKAGGLTGGLNFDSKVRRPSYTHEDLFYGFILGMDSFALGLIKAKALIADGRLDSFVKDRYASYGSGIGAKIRDHSATLEELAAYALAKDTVALPGSGRQEYLESIINQILFQ

SEQ ID NO: 3 - DNA, 21 bp, Artificial Sequence
  Synthetic DNA/primer
tgggaatattaccgctcgaag

SEQ ID NO: 4 - DNA, 29 bp, Artificial Sequence
  Synthetic DNA/primer
ctttaaaaaatttccaattttcctttacg

SEQ ID NO: 5 - DNA, 33 bp, Artificial Sequence
  Synthetic DNA/primer
ggaaattttttaaagtcgcagccacgggtcaac

SEQ ID NO: 6 - DNA, 50 bp, Artificial Sequence
  Synthetic DNA/primer
gtgaattgagtcattttttattattagtcttttttttttttgacaatatc

SEQ ID NO: 7 - DNA, 31 bp, Artificial Sequence
  Synthetic DNA/primer
atgactcaattcactgacattgataagctag

SEQ ID NO: 8 - DNA, 37 bp, Artificial Sequence
  Synthetic DNA/primer
ccttaaatcaacgtcatattctttattggctttatac

SEQ ID NO: 9 - DNA, 24 bp, Artificial Sequence
  Synthetic DNA/primer
gacgttgatttaaggtggttccgg

SEQ ID NO: 10 - DNA, 25 bp, Artificial Sequence
  Synthetic DNA/primer
atgtctgaaccagctcaaaagaaac

SEQ ID NO: 11 - DNA, 52 bp, Artificial Sequence
  Synthetic DNA/primer
agctggttcagacattttgaatatgtattacttggttatggttatatatgac

SEQ ID NO: 12 - DNA, 25 bp, Artificial Sequence
  Synthetic DNA/primer
actggtagagagcgactttgtatgc

SEQ ID NO: 13 - DNA, 41 bp, Artificial Sequence
  Synthetic DNA/primer
caaagtcgctctctaccagtcgctttcaattcatttgggtg

SEQ ID NO: 14 - DNA, 26 bp, Artificial Sequence
  Synthetic DNA/primer
tgtatatgagatagttgattgtatgc

SEQ ID NO: 15 - DNA, 39 bp, Artificial Sequence
  Synthetic DNA/primer
actatctcatatacaatggtcaaaccaattatagctccc

SEQ ID NO: 16 - DNA, 25 bp, Artificial Sequence
  Synthetic DNA/primer
aaatggatattgatctagatggcgg

SEQ ID NO: 17 - DNA, 41 bp, Artificial Sequence
  Synthetic DNA/primer
gatcaatatccatttcttggtgtgtcatcggtagtaacgcc

SEQ ID NO: 18 - DNA, 35 bp, Artificial Sequence
  Synthetic DNA/primer
agttttaattacaaaatggctgccggtgtcccaaa

SEQ ID NO: 19 - DNA, 36 bp, Artificial Sequence
  Synthetic DNA/primer
ttgtaattaaaacttagattagattgctatgctttc

SEQ ID NO: 20 - DNA, 18 bp, Artificial Sequence
  Synthetic DNA/primer
aggaacagccgtcaaggg

SEQ ID NO: 21 - DNA, 35 bp, Artificial Sequence
  Synthetic DNA/primer
ttgacggctgttcctcttcccttttacagtgcttc

SEQ ID NO: 22 - DNA, 32 bp, Artificial Sequence
  Synthetic DNA/primer
tttgttttgtgtgtaaatttagtgaagtactg

SEQ ID NO: 23 - DNA, 41 bp, Artificial Sequence
  Synthetic DNA/primer
tacacacaaaacaaaatgtctcaaatttttaaggatatccc

SEQ ID NO: 24 - DNA, 39 bp, Artificial Sequence
  Synthetic DNA/primer
agcgctcttactttagcgatcgcactagtttattgaaac

SEQ ID NO: 25 - DNA, 28 bp, Artificial Sequence
  Synthetic DNA/primer
taaagtaagagcgctacattggtctacc

SEQ ID NO: 26 - DNA, 36 bp, Artificial Sequence
  Synthetic DNA/primer
taacattcaacgctattactccgcaacgcttttctg

SEQ ID NO: 27 - DNA, 26 bp, Artificial Sequence
  Synthetic DNA
tagcgttgaatgttagcgtcaacaac

SEQ ID NO: 28 - DNA, 41 bp, Artificial Sequence
  Synthetic DNA
tttgtttgtttatgtgtgtttattcgaaactaagttcttgg

SEQ ID NO: 29 - DNA, 42 bp, Artificial Sequence
  Synthetic DNA/primer
acataaacaaacaaaatgttgtgttcagtaattcagagacag

SEQ ID NO: 30 - DNA, 39 bp, Artificial Sequence
  Synthetic DNA/primer
aaataatcggtgtcattagatgagagtcttttccagttc

SEQ ID NO: 31 - DNA, 25 bp, Artificial Sequence
  Synthetic DNA/primer
tgacaccgattatttaaagctgcag

SEQ ID NO: 32 - DNA, 18 bp, Artificial Sequence
  Synthetic DNA/primer
agagcgcgcctcgttcag

SEQ ID NO: 33 - DNA, 33 bp, Artificial Sequence
  Synthetic DNA/primer
aacgaggcgcgctctaattccgctgtatagctc

SEQ ID NO: 34 - DNA, 45 bp, Artificial
  Synthetic DNA/primer
ataatgtatgctatacgaagttatagggaaagatatgagctatac

SEQ ID NO: 35 - DNA, 43 bp, Artificial Sequence
  Synthetic DNA/primer
tatagcatacattatacgaagttatacgacatcgtcgaatatg

SEQ ID NO: 36 - DNA, 33 bp, Artificial Sequence
  Synthetic DNA/primer
tattaatttagtgtgtgtatttgtgtttgtgtg

SEQ ID NO: 37 - DNA, 34 bp, Artificial Sequence
  Synthetic DNA/primer
cacactaaattaataatgagccatattcaacggg

SEQ ID NO: 38 - DNA, 38 bp, Artificial Sequence
  Synthetic DNA/primer
tttagtagacatgcattacaaccaattaaccaattctg

SEQ ID NO: 39 - DNA, 37 bp, Artificial Sequence
  Synthetic DNA/primer
tgcatgtctactaaactcacaaattagagcttcaatt

SEQ ID NO: 40 - DNA, 54 bp, Artificial Sequence
  Synthetic DNA/primer
ataatgtatgctatacgaagttatgggtaataactgatataattaaattgaagc

SEQ ID NO: 41 - DNA, 47 bp, Artificial Sequence
  Synthetic DNA/primer
tatagcatacattatacgaagttattgacaccgattatttaaagctg

SEQ ID NO: 42 - DNA, 39 bp, Artificial Sequence
  Synthetic DNA/primer
attttactggctggagtatgctgcagctttaaataatcg

SEQ ID NO: 43 - DNA, 26 bp, Artificial Sequence
  Synthetic DNA/primer
tccagccagtaaaatccatactcaac

SEQ ID NO: 44 - DNA, 20 bp, Artificial Sequence
  Synthetic DNA/primer
gtctttttgccagccagtcc

SEQ ID NO: 45 - DNA, 33 bp, Artificial Sequence
  Synthetic DNA/primer
cacaccttcccccttgatcctctagagtcgacc

SEQ ID NO: 46 - DNA, 35 bp, Artificial Sequence
  Synthetic DNA/primer
gcggtaatattcccagatccccgggtaccgagctc

SEQ ID NO: 47 - DNA, 20 bp, Artificial Sequence
  Synthetic DNA
taacatgcggcatttcctgg

SEQ ID NO: 48 - DNA, 25 bp, Artificial Sequence
  Synthetic DNA/primer
tcacatccattccttgaattgattg

SEQ ID NO: 49 - DNA, 41 bp, Artificial Sequence
  Synthetic DNA/primer
aaggaatggatgtgaactagttttcttctttcctcctcttc

SEQ ID NO: 50 - DNA, 21 bp, Artificial Sequence
  Synthetic DNA/primer
cgttggttgaaacgttgtctg

SEQ ID NO: 51 - DNA, 38 bp, Artificial Sequence
  Synthetic DNA/primer
acgtttcaaccaacgaattccgctgtatagctcatatc

SEQ ID NO: 52 - DNA, 39 bp, Artificial Sequence
  Synthetic DNA/primer
ttaagagcgatttgtgtatgctgcagctttaaataatcg

SEQ ID NO: 53 - DNA, 32 bp, Artificial Sequence
  Synthetic DNA/primer
acaaatcgctcttaaatatatacctaaagaac

SEQ ID NO: 54 - DNA, 21 bp, Artificial Sequence
  Synthetic DNA/primer
atcttgccctattgcattccc

SEQ ID NO: 55 - DNA, 33 bp, Artificial Sequence
  Synthetic DNA/primer
gcaatggatacttctggggatcctctagagtcg

SEQ ID NO: 56 - DNA, 45 bp, Artificial Sequence
  Synthetic DNA/primer
aaatgccgcatgttagggtaccgagctcgaattcactggccgtcg